(19) United States
(12) Patent Application Publication
(10) Pub. No.: US 2002/0048822 A1
(43) Pub. Date: Apr. 25, 2002

(54) MARKING OF PRODUCTS WITH ELECTROACTIVE COMPOUNDS

(76) Inventors: JAMES H. RITTENBURG, PERKASIE, PA (US); ELAINE PEARS, ACOMB (GB); RICHARD TIER, CLIFTON MOOR (GB); STEPHEN P. TATTERTON, SOUTHBANK (GB); IAN ACWORTH, MELROSE, MA (US); PAUL GAMACHE, HUDSON, NH (US); WAYNE MATSON, AYER, MA (US)

Correspondence Address: PAUL T CLARK, CLARK & ELBING LLP, 176 FEDERAL STREET, BOSTON, MA, 02110

(21) Appl. No.: 09/404,414

(22) Filed: Sep. 23, 1999

(51) Int. Cl. G01N 33/543

(52) U.S. Cl. 436/518

(57) ABSTRACT

In general, the invention features a method of marking a product for identification in which a marker, composed of an electroactive compound, is added to the product and subsequently measured using an electrochemical detector.

### BACKGROUND OF THE INVENTION

This invention relates to the marking of products to establish their identity and source.

Major problems experienced in many areas of the world and in connection with many different products is that of product counterfeiting, unauthorized distribution and sale of a product (e.g., grey market trading, parallel trading, product diversion), as well as false liability based on product substitution.

Throughout the world, manufacturers provide the products they sell with a visually distinctive appearance, packaging or labels so that customers can distinguish their products from those of others. As a result, their customers learn to associate the visually distinctive appearance with certain standards of quality, and, if they are satisfied with those standards, will buy products provided with that visually distinctive appearance in preference to others. Once customers have acquired a preference for products provided with a particular visually distinctive appearance, the manufacturers become vulnerable to product counterfeiting.

A counterfeit product consists of a product that is provided with a visually distinctive appearance, or a brand name, confusingly similar to that of a genuine product. Customers seeing the visually distinctive appearance or the familiar brand name provided to the counterfeit product, buy this product in the expectation that they are buying a genuine product.

There are many ways known of providing products with a visually distinctive appearance. In general, the visually distinctive appearance is provided either directly to the product or to an article with which the material is associated, for example a label, wrapper or container. The visually distinctive appearance may be, for example, a distinctive shape or configuration, a distinctive marking, or a combination of the two. A particularly preferred visually distinctive appearance is a trademark.

The material of a counterfeit product may be the same as, or different from the material of a genuine product. Often the material of the counterfeit product is the same, but of inferior quality. For instance, it is usually difficult to distinguish a chemical product having a particular chemical formula and made by one manufacturer, from the same chemical, with the same formula, but made by a different manufacturers. This is particularly so if the two manufacturers use the same production process. For this reason, it is not difficult for the unscrupulous to establish the chemical formula of an active ingredient in a composition, and the relative amounts of the various ingredients in the composition, and then pass off his own product as that of another manufacturer.

In addition to product counterfeiting, product adulteration is another major problem. Product adulteration takes place when a product is tampered with such as by dilution. An example of such a problem lies in the adulteration of lubricating oils, or other petroleum based products such as fuels, by addition of a counterfeiter's oil or fuel, to a genuine product. Such adulteration is not only financially damaging to the manufacturer but the consequent lowering of performance which can occur can cause damage to the consumer and consequently harm the reputation of the genuine product. A method of overcoming this problem has been previously proposed involving the incorporation of a visible dye in the product. Such a strategy is easily copied.

WO 87/06383 discloses a method of labeling an item or substrate by means of macromolecules, in particular, DNA or proteins. European patents 0327163 and 0409842, and U.S. Pat. No. 5,429,952 disclose methods of marking products with chemicals that can be measured by immunoassay or by other specific binding assays.

U.S. Pat. Nos. 5,304,493, 5,244,808, and 4,918,020 disclose methods of marking petroleum products with dyes and subsequent detection of the dyes using standard solid phase extraction technology.

U.S. Pat. No. 5,474,937 discloses methods for marking chemicals using stable isotopes and detection of these markers using GC/MS.

U.S. Pat. No. 5,879,946 discloses methods for monitoring chemical additives using chemiluminescent markers and detection of these markers using a luminometer.

### SUMMARY OF THE INVENTION

The present invention, provides a novel means of marking products which provides substantial benefits over previously disclosed technologies. The invention uses electroactive compounds (described in greater detail below) as markers, thus enabling highly sensitive and selective quantitative measurement, and providing for a huge array of potential marker compounds, many of which are readily available, inexpensive, and generally regarded as safe (GRAS). Thus, these types of marker compounds are useful for marking ingested products such as pharmaceuticals and branded spirits, as well as very high volume products such as fuels.

Accordingly, the invention features a method of marking a product for identification in which an electroactive (electrogenic) compound is associated with the product as a marker, where the electroactive compound is non-deleterious to the product, inert with respect to the product, and not already associated with the product. For purposes of this application, an electrogenic compound is any compound that can undergo oxidation (loss of electrons) or reduction (gain of electrons) when subjected to a difference in electric potential. The invention provides a method of labeling a product in such a way that the presence of the marker can only be easily established by someone who knows the identity of the marker, but could not be routinely determined by a counterfeiter or other person unfamiliar with the marker. Thus, a counterfeit and a genuine product can be distinguished by the absence of the marker in the former and the presence of the marker in the latter.

The product is generally a commercial product and may be either solid, liquid, semisolid or gas. The marker may be added directly to the product (e.g., attached to a surface of the product or mixed with the product itself) or associated with a label, tag, or other product packaging material.

By “marking a product for identification” is meant associating a marker with a product so that the source, identity, or other information about the product including productions site, production date, batch, and shelf-life may be established. Identification of a marked product can also facilitate: 1) monitoring of manufacturing or other processes, including monitoring process streams and blending controls; 2) product monitoring for security or regulatory purposes, such as marking the source country of products for customs and marking regulated substances; 3) detecting and monitoring spillages of marked materials, including the detection of residues of marked products, such as pesticides, herbicides, fertilizers, toxic wastes, organic pollutants (such as TBT and dioxins) and other chemicals; 4) tracing a product, such as marking a process chemical to monitor the rate of addition of the chemical to a system (e.g., a water system) in order to optimize chemical dosage; and 5) studies of biodegradation of a compound, e.g. in soil biodegradation studies. Marking a product for identification also includes associating a product with a particular concentration of a marker, so as to facilitate the detection of product alduteration by way of dilution, concentration changes, or the addition of foreign substances.

The present invention allows the practitioner to, for the purposes of marking a product, develop or select the chemical structure (e.g., electroactive molecule) which will be specifically recognized at low concentrations by an electrochemical detector and which provides the required characteristics for a particular product marking application. Such required marker characteristics may include: (1) solubility or non-solubility in a product or solvent; such solubility or non-solubility can be important either for efficiently incorporating the marker into the product, or for extracting the marker for testing; (2) stability during extremes of temperature, pH or other physical or chemical conditions inherent in many manufacturing processes, (3) stability within a product or adherence to the surface of a product during conditions of use or storage, (4) regulatory acceptance for use in ingested products such as pharmaceuticals and spirits, or other regulated products such as agricultural chemicals.

The use of electroactive markers and electrochemical detection allows for highly sensitive and selective measurement of intentionally added electroactive marker compounds. Using electrochemical detection in conjunction with liquid chromatography provides an effective means for detecting and decoding multiple marker combinations and concentrations.

Other features and advantages of the invention will be apparent from the following description of the preferred embodiments thereof, and from the claims.

### BRIEF DESCRIPTION OF THE FIGURES

FIG. 1 is a chromatogram for unmarked regular grade gasoline illustrating the lack of any electroactive peaks.

FIG. 2 is a chromatogram for unmarked diesel fuel illustrating the lack of any electroactive peaks.

FIG. 3 is a chromatogram for marked regular grade gasoline illustrating a well defined peak of electrochemical activity.

FIG. 4 is a chromatogram for marked diesel fuel illustrating a well defined peak of electrochemical activity.

### DETAILED DESCRIPTION

Markers

The marker of the invention is capable of being detected by an electrochemical detector. The marker should be compatible, i.e., non-deleterious, with the product which it marks. The marker may be comprised of any electroactive chemical. Electroactive chemicals are characterized by their ability to gain electrons (reduction) or lose electrons (oxidation) when subjected to a difference in electric potential. Preferably the marker will be non-toxic if used in a manner in which it is intended to be ingested. Preferably the marker is visually undetectable when present in the product.

The marker should in general be one which is not normally present in the chemical or composition; for example, it is not a by-product of the production process, normal impurity, or standard additive for that chemical, or chemical composition. In preferred embodiments, the marker compound is present in very low concentrations, e.g., in the order of parts per million or parts per billion. The marker is preferably inert with respect to the product in the sense that it does not react with the product that it labels. Moreover, the ability to detect the marker should not be adversely affected by interaction with the product or compound it labels. For example, where the marker is to be detected by an electrochemical detector coupled to an HPLC, the marker must exhibit some combination of unique retention time and unique range of voltage potential over which electron transfer occurs.

Depending on the specific application, certain criteria must be considered in selecting an appropriate chemical compound that is capable of performing acceptably as a marker. Most importantly, the compound must possess a specific molecular moiety, which is electroactive. Exemplary marker compounds may typically include, flavenoids and phenolics (e.g., eugenol, hesperidin, umbelliferone, etc.), polynuclear aromatics (e.g., azobenzene, 2-aminobiphenyl, 1-aminoanthracene, etc.), amino acids (e.g., glycine, taurine, alanine, etc.), and antioxidants (e.g., 2-t-butylphenol, octyl gallate, 2,5,-di-t-butylhydroquinone).

It will be appreciated that a wide range of compounds are suitable as marker compounds so long as they are compatible with and non-deleterious to the product being marked. Thus the use of oil-compatible, water-compatible and solids-compatible or food grade compounds as marker compounds is envisaged dependent on the product being marked.

Products for marking

It will be appreciated that the marker compound may be associated with the product in a wide variety of ways. Thus the marker compound may be present in or on all or part of the product, or in or on all or part of a label, wrapper, container or other packaging material associated with the product. The marker compound is usually mixed with the product, but may alternatively be present independently of the product, for example the marker may be present in the product packaging or labeling, or printed onto the surface of the product or product packaging.

The product marked may be solid, semi-solid, fluid, or gas.

Examples of solid products include pharmaceutical tablets, capsules, and powders; solid formulations of agrochemicals such as insecticides, herbicides, fungicides and fertilizers; polymers, plastics, and rubbers; textiles such as clothing; designer or specialty products such as crystal, china, and silver goods; original works of art such as paintings and sculptures, recordings such as gramophone records, tape cassettes, floppy discs and compact discs; electrical goods such as television sets, computers and radios; motor vehicle components and cameras; paper such as documents, confidential papers, notes, securities, labels, and packaging; chemical products such as biocides, cosmetics such as creams; and food products.

Examples of fluid products include oil-based products such as lubricating oils, gasoline, diesel and liquified petroleum products; paints and coatings; perfumes; cosmetics; inks; drinks such as wine, whisky, sherry, gin and vodka; liquid pharmaceutical formulations such as syrups, emulsions, and suspensions; liquid agrochemical formulations; chemical compositions; and industrial solvents. The fluid product is preferably liquid. One preferred class of products encompasses oil based products such as lubricating oils and fuels.

Examples of gases include stack emissions (e.g., for pollution tracing), air parcels (e.g., for study of weather patterns),air samples within storage containers (e.g., to ensure that such containers have not been opened) and chlorofluorocarbons (e.g., diflurodichloromethane, tetrafluoromethane, octafluorocyclobutane, trichlorofluoromethane, etc.) used in aerosol propellants, air conditioning and refrigeration.

When the product is a liquid, the marker compound is preferably colorless at concentrations present in the marked product and soluble in the liquid product so that its presence can only be detected by subsequent assay. It is preferably also odorless at marker concentrations present in the marked product.

Preferably only trace quantities of marker are used. Typically a marker compound will be incorporated with a product at a concentration in the range of from 1 part per billion (ppb) to 25 parts per million (ppm). Preferably the concentration will be in the range of from 20 ppb-2 ppm.

The ability to detect concentrations of marker compound at very low concentrations, i.e., in the parts per billion range, is a particular advantage of the method according to the invention. Thus only small quantities of marker compound need to be used.

Another particular advantage of the invention is that the use of electrochemical detection can provide very selective detection of intentionally added electroactive compounds within complex sample matrices. For example, fuels and lubricants contain a wide array of aromatic and polyaromatic compounds that absorb and fluoresce across the UV and visible regions of the electromagnetic spectra, greatly limiting one's ability to measure low analyte concentrations based on the use of standard fluorometric and spectrophotometric detectors. However, electrochemical detection of appropriately selected markers can be accomplished with virtually no matrix background effects from other fuel components due to the extreme selectivity provided by electrochemical detectors. Because of the chemical complexity of fuel and lubricant matrices, it would be unexpected to find an analytical method that could selectively and accurately measure trace levels of marker without extensive sample clean-up. Even with extensive sample clean-up, the detection sensitivity possible with other technologies is not likely to match the sensitivity of electrochemical detection for electroactive compounds..

Preferably several markers are included in chemical or chemical composition products. The ratios of the concentrations of the markers in each chemical or composition labeled are then preferably unit ratios, e.g., in the case where there are two markers the ratio of concentration of one to that of the other may be 1:1, 1:2, 1:3, 1:4, etc. The total amount of marker compound(s) added is such that each marker compound is preferably added at a level of not more than 10 parts per million, and more preferably at not more than 100 parts per billion (by weight). Use of multiple markers can impart batch or manufacturing site specific information into a product, such as a pharmaceutical tablet or a branded spirit.

In one embodiment, a plurality of markers are present that possess common chemical characteristics such that they can be separated and resolved using a single chromatographic method. The plurality of markers can subsequently be resolved using a combination of retention time and electrochemical activity. Markers with identical or overlapping retention times can also be resolved and accurately measured purely by differences in their electrochemistry (e.g., the range of potential voltage difference at which oxidation or reduction occurs). In this way complex codes can be imparted to products through the use of multiple marker combinations and concentrations.

Marker detection

The markers may be detected in a sample of the product either qualitatively or quantitatively. Quantitation of the marker in a product facilitates detection of product adulteration by dilution of the original product. Quantitation of the marker can also be used to decode various marker concentrations and ratios that provide information such as manufacturing site, manufacturing date, batch, country of origin, etc.

Quantitation of the marker can also be used in assessing the physical parameters of fluid systems. For example, one can mark a known volume of a liquid (e.g., water) at a known concentration, add this marked sample to a fluid system of unknown volume, disperse the marked sample in the fluid system, assay a sample from the fluid system, and calculate the dilution effect to determine the volume of the fluid system.

The marker compound can be incorporated with the product in an aqueous or non-aqueous medium, and an assay to detect the marker may be carried out directly on a sample thereof. The sample may be filtered to remove solids, if necessary.

In general, producing a sample of a product to assay for a marker will comprise one or more steps selected from extraction of the marker compound from the product; dilution of the product with an aqueous or an organic solvent; filtration; evaporation; precipitation; and solid phase extraction or separation of the marker compound(s), e.g., liquid chromatography (e.g. reverse phase, normal phase, ion-exchange) using various type of packed columns containing resins such as silica or functionalized silica particles.

The solvent chosen for extracting the marker compound from the product prior to assay naturally depends on the natures of the product and the marker. Depending upon the natures of the product and the marker, the solvent will in general comprise one or more of water; hydrocarbons, for example benzene, toluene, xylene, hexane, heptane and octane; sulphoxides, for example dimethylsulphoxide; halogenated hydrocarbons; chlorinated solvents, for example, chlorobenzene, methylene chloride, chloroform and carbon tetrachloride; ethers, for example diethyl ether, dioxane and tetrahydrofuran; amides, for example dimethylformamide and dimethylacetamide; nitrites, for example, acetonitrile; alcohols, for example methanol, ethanol and propanol; esters, for example ethyl acetate; and ketones, for example acetone. Optionally the extraction solvent may also comprise buffer salts such as Tris buffer (Tris[hydroxymethyl]amino-methane). The solvent system used preferably yields the extracted marker compound in a liquid phase suitable directly for the subsequent detection assay. Obviously, in some cases where the marked sample is a liquid, no sample preparation or extraction will be required.

The present invention facilitates the identification of several different batches of a product (e.g., a chemical or chemical composition) by the use of a single marker compound. This is because a single marker compound may be employed in different concentrations in different batches and each batch identified by determination of the concentration of the marker in that batch.

In certain preferred embodiments a plurality of markers are included in a chemical or composition. In this case the number of possible permutations of concentration and markers is increased and batches may be identified with increased certainty by measuring relative concentrations of the markers.

Marker measurement by electrochemical detection

When certain compounds are subjected to a potential difference they undergo molecular rearrangement at the working electrodes' surface with the loss (oxidation) or gain (reduction) of electrons. Such compounds are said to be electroactive and undergo electrochemical reactions. The most common form of electrochemical detector is the amperometric detector in which the voltage potential is kept constant and the current produced from the electro-chemical reaction is measured. This is known as potentiostatic amperometry.

For applications in analytical chemistry the preferred use of the electrochemical detector is in conjunction with an HPLC system to analyze eluent from the HPLC column. The HPLC system facilitates the separation of various compounds within a complex sample matrix based on the retention times exhibited by the individual compounds when passing through the chromatographic column. Retention time for individual compounds are a function of the type of column packing employed, the physicochemical properties of the compound, and the solvent system used to transport the sample through the chromatography column. The electrochemical detector provides a major advantage over the spectrophotometric detectors (ultraviolet-visible) that are commonly used, due to its exquisite selectivity and sensitivity. The electrochemical detector at a specific voltage potential is blind to many compounds in the sample making chromatographic separation and peak identification easier than with spectrophotometric detectors. For the present application, specific sample matrices can initially be characterized to determine where (e.g., what ranges of voltage potential) the “blind” spots are and then marker selection can be made rationally, based on the electrochemical characteristics of candidate marker compounds. The sensitivity of electrochemical detectors is also a major advantage over spectrophotometric techniques with electrochemical detectors generally demonstrating 10 to 500 fold improvements in detection sensitivity.

A preferred design of electrochemical detector used in conjunction with HPLC is the flow through or porous amperometric detector in which the column eluent passes through the graphite working electrode (Ian Acworth and Paul Gamache, Amercian Laboroatory, May 1996). With this design, the surface area of the working electrode is large, and close to 100% of the marker will react, and thus no signal is wasted. When the efficiency of detection is 100%, this is referred to as coulometry and these specialized amperometric detectors are termed coulometric detectors. Coulometrically efficient sensors have a number of practical advantages in selectivity and sensitivity, making them ideal for use in an electrode array configuration. Since the coulometric electrodes effectively measure 100% of the concentration of the analyte, they remove the analytes signal from sensors further along the array. This allows coeluting compounds to be effectively resolved even if their half wave potentials (the potential at half signal maximum) differ by only 60 mv. A preferred embodiment of this invention is the use of coulometric electrode arrays for detection of intentially added electroactive markers. Other electrochemical detection systems having features that can be used in the invention are described in U.S. Pat. Nos. 4,233,031, 4,404,065, and 4,511,659 which are hereby incorporated by reference.

Detection of Marker(s) using Combined Technologies

As described in the previous sections, the use of electroactive markers and electrochemical detectors linked with HPLC provides for a powerful marking and detection system that enables complex coding through the use of multiple markers and marking concentrations. Other marking technologies such as the binding pair technology described in U.S. Pat. No. 5,429,952 provide for simple but highly sensitive and specific field methods for detection of intentionally added markers. It is anticipated that the combination of a simple field test, such as an immunoassay, to indicate that a product is marked and contains an underlying code; along with a laboratory method, such as HPLC equipped with electrochemical detectors capable of decoding marker combinations and concentrations, will provide additional utility for certain marking applications. For example, a pharmaceutical product such as a tablet could be marked to indicate authenticity as well as to identify specific manufacturing sites. Use of a qualitative field immunoassay specific for one marker that would always be incorporated into the tablet provides for a quick and simple means of testing product for authenticity and for indicating that a particular sample should contain an underlying code that could be deciphered in the laboratory using HPLC with electochemical detection. Using a total of three different markers at three different concentrations (with one marker always present for detection with a binding pair assay) would allow for 48 possible codes to be incorporated into the pharmaceutical tablet. This information could be used to identify manufacturing sites and for identifying and tracking product diversion (parallel trading) activities. It can be appreciated that increasing the number of markers and marking combinations will dramatically increase the possible number of different codes that can be incorporated into the product. In one embodiment the complementary binding pair (e.g. antibody) could recognize a marker that was also electroactive. However, this is not a necessity for the combination system to still provide substantial value. This approach could applied to many different industries for example in the petroleum sector for identification of particular refineries or in the spirits and beverage industries for identification of specific bottling plants.

The invention will now be further described with reference to the following examples.

### EXAMPLE 1

Marking a Pharmaceutical Matrix with an Electroactive Compound

A 12% w/w pharmaceutical tablet coating suspension was marked with the electroactive compound folic acid at a concentration of 5.33 μg/g. A marked and unmarked sample was analyzed in the following manner:

A 0.4 g aliquot of each of the marked and unmarked Pharmaceutical suspensions were added to 0.6 ml volumes of sodium hydrogen carbonate (0.1 M) and then diluted into 9 ml volumes of water. After mixing, the samples were filtered through a 0.2 μm acrodisc syringe filter. Fifty microliters of each of the filtered samples were injected onto into the HPLC system. A Gilson HPLC System 1, Applied Biosystems absorbance detector, and ESA Inc.8 electrode coulometric array detector were used along with a Capitol 10 cm C-18 reverse phase ODS2 HPLC column. An isocratic mobile phase consisting of 3.5% acetonitrile in aqueous phosphate buffer pH 6.75 was used to transport the sample through the HPLC column. The identity of peak due to folic acid was confirmed using a UV detector in series with the coulometric array detector. An applied voltage of 780 mV with a single electrode enabled the greatest detection sensitivity of folic acid. Using standard solutions prepared in water the sensitivity of the coulometric array detector was shown to be approximately 100 times better than spectrophotometric detection at the λ-max of 284 nm. Folic acid could be detected at concentrations down to 2.3 ng/ml when using a 50 μl injection volume.

Excellent chromatography was obtained from the analysis of the marked tablet coating sample, with the only peak detected electrochemically being folic acid. The actual amount of folic acid detected was 5.11 μg/g which compared very favorably to the theoretical concentration of 5.33 μg/g (96% recovery). No folic acid was detected in the unmarked sample.

### EXAMPLE 2

Marking a Spirit with an Electroactive Compound

A commercial brandy was marked with the electroactive compound folic acid at a concentration of 200 ng/ml. The marked sample was analyzed in the following manner:

Ten milliliters of marked and unmarked brandy were rotary evaporated down to 3 ml, then transferred to a 10 ml volumetric flask and made up to volume with deionized water. Fifty microliters of each solution was injected onto the HPLC. The same set of chromatographic conditions as detailed in Example 1 were used for the analyses described in this example.

Folic acid was readily detected in the marked brandy by the ESA Coulometric Electrode Array detector using an applied voltage of 780 mv. The analysis indicated the presence of approximately 133 ng/ml of marker. No folic acid was detected in the unmarked sample.

### EXAMPLE 3

Marking Fuel with an Electroactive Compound

Gasoline and diesel fuel were obtained from commercial sources and marked at 2 μg/ml with the electroactive compound α-tocopherol. Marked and unmarked fuel samples were analyzed in the following manner:

Marked and unmarked diesel and gasoline samples were diluted 1:10 in methanol prior to injection of 10 μl of the diluted samples onto the HPLC. The apparatus used for analysis included a CoulArray® Model 5600 HPLC detection system comprised of one Model 580pump, PEEK® pulse damper, Model 540 autoinjector, CoulArray® thermostatic chamber, serial array of eight coulometric electrodes and CoulArray® for Windows® software. (ESA Inc. Chelmsford, Ma.)

The chromatography column used was a 150×4.6 mm i.d., 5 μ, C18 maintained at a temperature of 37° C. The mobil phase was methanol-water, 95:5 (v/v) containing 20 mM ammonium acetate, pH4.4. The flow rate was 2.0 ml/min and the detector potentials used were 0, 100, 200, 300, 400, −200, −300 (mv vs. Pd). Analysis time was 10 minutes.

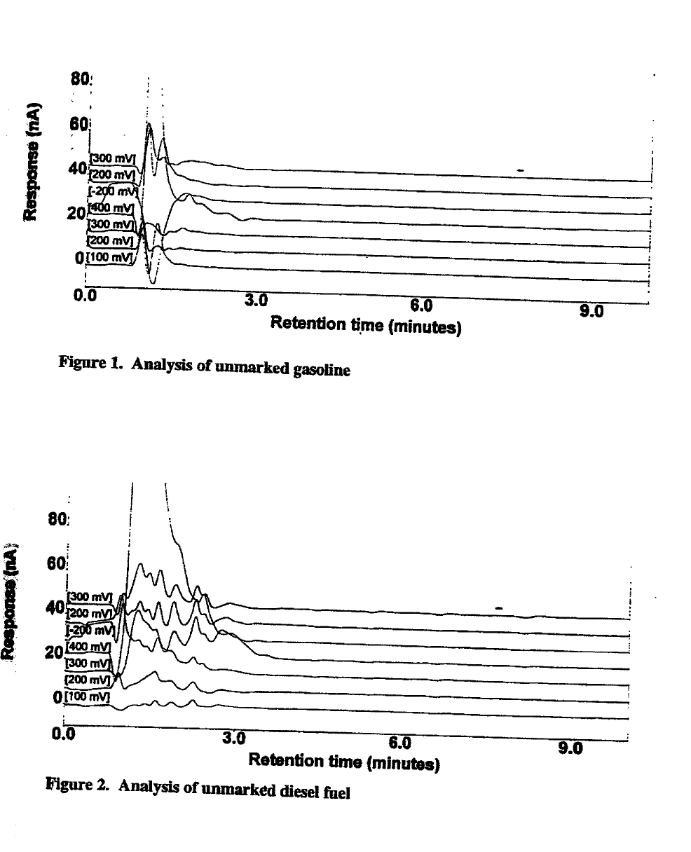

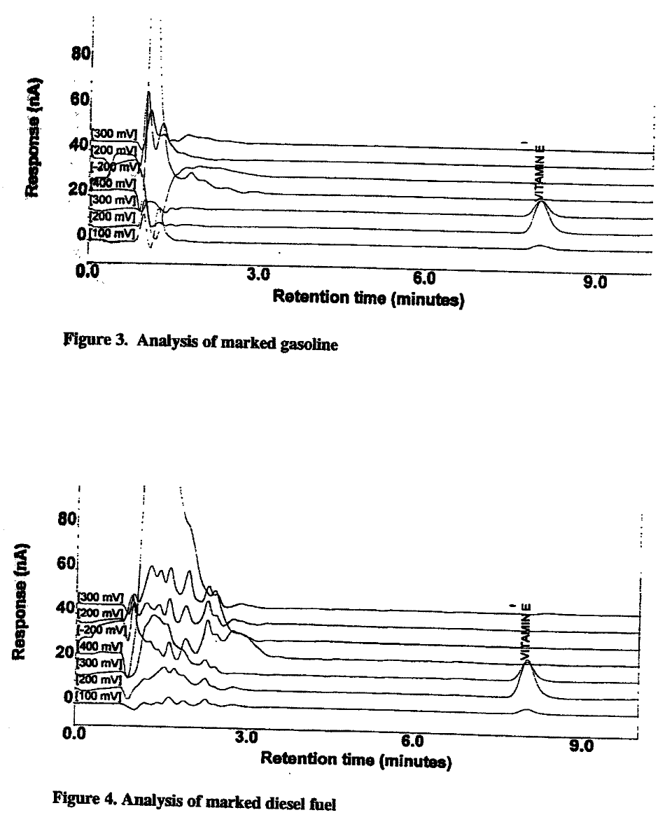

The chromatograms obtained are shown in FIGS. 1, 2, 3, and 4. FIGS. 1 and 2 show that for unmarked gasoline and diesel, respectively, there is no background interferences that occur across the voltage ranges utilized. This is illustrated by the virtually flat baselines that were observed. FIGS. 3 and 4 clearly show that the electroactive marker,_α-tocopherol, can easily be detected and quantified in gasoline and diesel, respectively. For the gasoline marked at a theoretical level of 2 ppm (μg/ml) an analytical result of 1.98 ppm was obtained (99% recovery). For the diesel sample marked at a theoretical level of 2 ppm and analytical result of 2.34 ppm was obtained (117% recovery). The very clean peaks observed for the α-tocopherol, in the 1:10 dilutions of gasoline and diesel fuel illustrate the exquisite selectivity of the electrochemical detector.

The lower limit of detection in this example was 50 ppb and was estimated using a signal to noise ratio of 3:1 in unmarked samples. Since only a 10 μl injection volume was utilized for these examples it is likely that even substantially lower detection levels would be possible with larger injection volumes. This example clearly shows the utility and power of this technology to detect intentionally added markers in fuels.

What is claimed is:

1. A method of marking a product and subsequently detecting the mar in the product as a means of identifying the product, said method comprising the steps of:
a) associating an electroactive compound as a marker with the product; and
b) detecting the marker in the product at a later point in time, as a means of identifying the product, using electrochemical analysis.

2. The method of claim 1, wherein the electrochemical analysis is performed on eluent from a chromatographic system.

3. The method of claim 2 wherein the chromatographic system is HPLC.

4. The method of claim 1 wherein the electrochemical detection is performed using one or more coulometric electrodes.

5. The method of claim 1 wherein the electrochemical detection is performed using amperometric electrodes.

6. A method of marking a product, and subsequently detecting the marker in the product, said method comprising the steps of:
a)associating with said product a first marker which is a member of a specific binding pair and a second marker that is detectable by electrochemical analysis;
b) detecting the first marker using a specific binding partner; and
c) detecting or measuring the second marker by electrochemical analysis.

7. The method of claim 6 wherein the electrochemical analysis is performed on eluent from a chromatographic system.

8. The method of claim 7 wherein the chromatographic system is HPLC.

9. The method of claim 6 wherein the electrochemical detection is accomplished using one or more coulometric electrodes.

10. A marked product comprising:
a) commercial petroleum product having associated with it; and
b) an electroactive marker not normally associated with said petroleum product.

11. A marked product comprising:
a) commercial pharmaceutical product having associated with it; and
b) an electroactive marker not normally associated with said pharmaceutical product.

12. A marked product comprising:
a) commercial spirits product having associated with it; and
b) an electroactive marker not normally associated with said spirits product.

13. The method of claim 6 in which the product marked is a commercial petroleum product.

14. The method of claim 6 in which the product marked is a commercial pharmaceutical product.

15. The method of claim 6 in which the product marked is a commercial spirits product.
